SEQUENCE LISTING (2 sequences)

SEQ ID NO: 1 - protein, 40 aa, Artificial Sequence
  leucine zipper peptide
RIARLEEKVKTLKAQNSELASTANMLREQVAQLKQKVMNY

SEQ ID NO: 2 - protein, 40 aa, Artificial Sequence
  leucine zipper peptide
LTDTLQAETDQLEDKKSALQTEIANLLKEKEKLEFILAAY

(19) United States
(12) Patent Application Publication
(10) Pub. No.: US 2010/0004134 A1
(43) Pub. Date: Jan. 7, 2010

(54) Combinatorial libraries of conformationally constrained polypeptide sequences

(76) Inventors: Lawrence Horowitz, Atherton, CA (US); Ramesh R. Bhatt, Belmont, CA (US); Aaron L. Kurtzman, San Carlos, CA (US)

Correspondence Address: Goodwin Procter LLP;Attn: Patent Administrator, 135 Commonwealth Drive, Menlo Park, CA, 94025-1105, US

(21) Appl. No.: 12/008,567

(22) Filed: Jan. 11, 2008

Related U.S. Application Data
(60) Provisional application No. 60/884,832, filed on Jan. 12, 2007.

(51) Int. Cl. C40B 20/00; C40B 40/10; C40B 40/02; C40B 40/06; C40B 30/04; C07H 21/00; C12N 15/63; C12N 1/00; C12N 5/00; C12N 1/21; C12N 1/19

(52) U.S. Cl. 506/2; 506/18; 506/14; 506/16; 506/9; 536/23.53; 435/320.1; 435/243; 435/325; 435/252.3; 435/254.2; 435/252.33

(57) ABSTRACT

The present invention concerns combinatorial libraries of conformationally constrained polypeptide sequences and their uses. In particular, the present invention concerns combinatorial libraries of conformational epitopes and their uses.

### CROSS-REFERENCE TO RELATED APPLICATION

This is non-provisional application filed under 37 CFR 1.53(b), claiming priority under USC Section 119(e) to provisional Application Ser. No. 60/884,832, filed Jan. 12, 2007.

### FIELD OF THE INVENTION

The present invention concerns combinatorial libraries of conformationally constrained polypeptide sequences and their uses. In particular, the present invention concerns combinatorial libraries of conformational epitopes and their uses.

### BACKGROUND OF THE INVENTION

The need to define the binding sites of monoclonal antibodies has led to the development of epitope libraries. Thus, Parmley and Smith, Gene 73:305 318 (1988), developed a bacteriophage expression vector, which could be used to construct large collections of bacteriophage, displaying short peptide sequences on their surface. Phage displaying foreign epitopes could then be purified by biopanning, as described, for example, by Parmley and Smith, supra; Cwirla, et al., Proc. Natl. Acad. Sci. USA 87:6378 6382 (1990); Scott & Smith, Science 249:386 390 (1990); Christian, et al., J. Mol. Biol. 227:711 718 (1992); Smith & Scott, Methods in Enzymology 217:228 257 (1993). This technique was subsequently extended to the identification of peptide ligands for antibodies by biopanning epitope libraries, which could be used, for example, in vaccine development and epitope mapping (Scott, J. K., Trends in Biochem. Sci. 17:241 245 (1992).

The known approaches for biopanning of epitope libraries have resulted in the identification of short (usually up to about 6 amino acids) linear epitope sequences, or peptide sequences, which do not occur within a native protein sequence but rather mimic a native linear epitope. Linear epitopes, however, are fragments of discontinuous or conformational epitopes, and have lower functional potencies than the conformational epitopes of which they are part. It would, therefore, be desirable to display conformational epitopes, or to mimic the essential properties of conformational epitopes by proximal placement of discontinuous epitopes that adopt or approximate conformation due to their proximity or the presence of a structural support element. Such conformational epitope libraries could include a physically selectable display of all conformational epitopes, be used to select antibodies to all conformational epitopes, and would have numerous additional benefits and utilities.

### SUMMARY OF THE INVENTION

The present invention is based, at least in part, on the recognition that proximal placement of discontinuous epitopes and/or the use of structural support elements can regenerate the essential properties of conformational epitopes, and that a similar approach can be extended to the regeneration of other three-dimensional structural or functional elements of proteins, such as ligand-binding regions of receptors, substrate-binding regions of enzymes, and the like.

Thus, in one aspect, the invention concerns a physically selectable display comprising tandem or multimeric assemblies of discrete or random fragments of one or more native or variant polypeptides, or sequences mimicking such fragments, wherein at least some of the assemblies form conformationally constrained polypeptide targets, and wherein at least some of the fragments are other than antibody fragments.

In another aspect, the invention concerns a screening method, comprising

(a) providing a physically selectable display comprising tandem or multimeric assemblies of discrete or random fragments of one or more native or variant polypeptides, or sequences mimicking such fragments, wherein at least some of such assemblies form conformationally constrained polypeptide tar-gets, and wherein at least some of the fragments are other than antibody fragments;

(b) contacting the display with a library of candidate binding partners under conditions wherein the conformationally constrained polypeptide targets and the candidate binding partners that have binding affinities to each other form target-binding partner complexes, and

(c) detecting at least some of the target-binding partner complexes formed.

In one embodiment, the method may comprise the additional step of (d) identifying the target sequences participating in the formation of at least some of the target-binding partner complexes detected.

In another embodiment, the target sequences participating in the formation of all target-binding partner complexes detected are identified.

In yet another embodiment, the candidate binding partners are antibodies, antibody fragments or antibody mimics.

In a further embodiment, the antibody, antibody fragment or antibody mimic sequences participating in the formation of at least some of the target-binding partner complexes are additionally identified.

In a still further embodiment, the foregoing method further comprises the step of enriching and segregating the target sequences and the antibody, antibody fragment or antibody mimic sequences participating in the formation of at least some of the target-binding partner complexes prior to step (d).

In an additional embodiment, the method further comprises the step of independently recovering the target sequences and the antibody, antibody fragment or antibody mimic sequences participating in the formation of at least some of the target-binding partner complexes following the enrichment and segregation and prior to step (d).

In a different embodiment, the target sequences participating in the formation of at least some of the target-binding partner complexes are parts of a conformational epitope.

In a further aspect, the invention concerns a method, comprising

(a) providing a physically selectable display comprising tandem or multimeric assemblies of discrete or random fragments of one or more native or variant polypeptide, or sequences mimicking such fragments, wherein at least some of the assemblies form conformational epitopes;

(b) contacting the display with an antibody library under conditions wherein the conformational epitopes and members of the antibody library that have binding affinities to each other form conformational epitope-antibody complexes; and

(c) detecting at least some of the conformational epitope-antibody complexes formed.

In a particular embodiment, the conformational epitopes are obtained by the expression of tandem or multimeric assemblies of gene fragments or their mimics.

In another embodiment, the gene fragments originate from a targeted, biologically relevant source, where the targeted, biologically relevant source may, for example, be selected from the group consisting of cells, tissues, organs and organisms. Other biologically relevant sources include stem cells, activated immune cells, diseased tissues, organs and pathological organisms.

In a further embodiment, at least some of the gene fragments are identified by analysis of gene expression data in a targeted, biologically relevant source.

The following specific embodiments apply to all aspects of the invention.

In all aspects, the preferable physically selectable display is a conformational epitope library.

In various embodiments, the display may contain tandem or multimeric assemblies of discrete or random fragments of more than one native or variant polypeptide, or sequences mimicking such fragments.

In other embodiments, at least some of the tandem or multimeric assemblies comprises two or more sequences from different parts of the same polypeptide, where the sequences may include intracellular sequences. In a particular embodiment, each of the tandem or multimeric assemblies comprise two or more sequences from different parts of the same polypeptide, where the sequences may include intracellular sequences.

In a further embodiment, the tandem or multimeric assemblies comprise an antibody or fragments of an antibody, and a ligand for the antibody or antibody fragment.

In further embodiments, in the tandem or multimeric assemblies, at least some of the fragments or mimicking sequences are directly fused to each other.

In different embodiments, in the tandem or multimeric assemblies, at least some of the fragments or mimicking sequences are coupled by an exogenous connecting sequence.

In additional embodiments, in the tandem or multimeric assemblies, at least some of the fragments or mimicking sequences consist of or comprise a structural support element, which, may for example, be a motif characteristic of one or more protein families, such as a helical bundle, β-sheet structure, trifoil structure, a membrane-spanning helix, or an extracellular loop.

In another embodiment, at least some of the conformationally constrained polypeptide targets are formed as a result of the proximity of the fragments present in said tandem or multimeric assemblies.

Alternatively, or in addition, at least some of the conformationally constrained polypeptide targets may be formed as a result of the presence of structural support elements in said tandem or multimeric assemblies.

In all aspects, the conformationally constrained polypeptide targets and/or the candidate binding partners may be displayed using a suitable display system, including, without limitation, viral, eukaryotic and bacterial and in vitro display systems, such as, for example, bacteriophage, mammalian, yeast, bacterial cell and spore display systems, ribosome, mRNA and DNA displays. In a particular embodiment, the spore display system is a Bacillus subtilis or Bacillus thuringiensis spore display.

In all aspects, the antibody fragments may be, without limitation, Fab, Fab′, F(ab′)₂, scFv, (scFv)₂, and dAb fragments, linear antibodies, single-chain antibody molecules, minibodies, diabodies, or multispecific antibodies formed from antibody fragments, and the antibody mimics may, for example, be affibodies or aptamers.

The present invention further concerns methods and means for making the physically selectable displays herein, including, without limitation appropriate coding sequences, vectors, and recombinant host cells.

### BRIEF DESCRIPTION OF THE DRAWINGS

FIG. 1 illustrates two approaches for presenting conformational epitopes in the displays of the present invention. In the first approach (Type I), two polypeptide fragments are connected to each other by a flexible linker and tethered to a display vehicle. In this case, formation of a conformational epitope is facilitated by the proximity of the two fragments, and the ability to fold, as a result of the flexible connecting linker. In the second approach (Type II) the two polypeptide fragments are connected by a structural scaffold and tethered to a display vehicle, where formation of a conformational epitope is facilitated by the structural scaffold.

FIG. 2 illustrates a method for simultaneous selection of conformational epitope and antibody libraries, where the conformational epitope library is presented using spore display while the antibody library is a phagemid library.

FIG. 3 illustrates an erythropoietin (EPO) crossover loop, a thrombopoietin (TPO) crossover loop and an EPO/TPO C-D crossover loop chimeric construct, which can be used to present conformational epitopes of EPO and/or TPO.

FIG. 4 illustrates the identification of conformational epitope-directed antibodies against thrombopoietin (TPO) using the EPO/TPO C-D crossover loop chimeric construct shown in FIG. 3, followed by selection on a native TPO crossover loop, also shown in FIG. 3.

FIG. 5 illustrates ligand-induced stabilization of conformational epitopes, using tethered antigen-antibody display.

FIG. 6 illustrates a method for simultaneous selection of conformational epitope and antibody libraries, where both libraries are presented using phage display.

### DETAILED DESCRIPTION OF THE INVENTION

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “epitope” as used herein, refers to a sequence of at least about 3 to 5, preferably at least about 5 to 10, or at least about 5 to 15 amino acids, and typically not more than about 500, or about 1,000 amino acids, which define a sequence that by itself, or as part of a larger sequence, binds to an antibody generated in response to such sequence. An epitope is not limited to a polypeptide having a sequence identical to the portion of the parent protein from which it is derived. Indeed, viral genomes are in a state of constant change and exhibit relatively high degrees of variability between isolates. Thus the term “epitope” encompasses sequences identical to the native sequence, as well as modifications, such as deletions, substitutions and/or insertions to the native sequence. Generally, such modifications are conservative in nature but non-conservative modifications are also contemplated. The term specifically includes “mimotopes,” i.e. sequences that do not identify a continuous linear native sequence or do not necessarily occur in a native protein, but functionally mimic an epitope on a native protein. The term “epitope” specifically includes linear and conformational epitopes.

As used herein, the term “conformational epitope” refers to an epitope formed by discontinuous portions of a protein having structural features of corresponding sequences in the properly folded full-length native protein. The length of the epitope-defining sequence (the sequence including the discontinuous portions making up the conformational epitope) can greatly vary as these epitopes are formed by the three-dimensional structure of the protein. Thus, amino acids defining the epitope can be relatively few in number, widely dispersed along the length of the molecule, being brought into correct epitope conformation via folding. The portions of the protein between the residues defining the epitope may not be critical to the conformational structure of the epitope. For example, deletion or substitution of these intervening sequences may not affect the conformational epitope provided that the sequences critical to epitope conformation are maintained. Thus, a “conformational epitope,” as defined herein, is not required to be identical to a native conformational epitope, but rather includes conformationally constrained structures that regenerate (exhibit) essential properties (such as qualitative antibody-binding properties) of native conformational epitopes.

“Linear epitopes” are fragments of discontinuous or conformational epitopes.

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J.

The phrase “conformationally constrained polypeptide target” refers to a binding sequence formed by discontinuous portions of a protein, or artificial sequences not occurring in a native protein, having structural features of corresponding sequences in the properly folded full-length native protein. The term specifically includes conformational epitopes, but also binding regions (pockets) of receptors, enzymes and other proteins, including sequences mimicking such binding regions. In all instances, the sequence defining the conformationally constrained polypeptide target can greatly vary as these conformationally constrained binding regions are formed by the three-dimensional structure of the protein. Thus, amino acids defining the conformationally constrained polypeptide target can be relatively few in number, widely dispersed along the length of the molecule, being brought into correct conformation via folding. The portions of the protein between the residues defining the binding region may not be critical to the conformational structure. For example, deletion or substitution of these intervening sequences may not affect the conformational binding region provided sequences critical to the proper conformation are maintained. Thus, a “conformationally constrained polypeptide target,” as defined herein, is not required to be identical to a structural element present in a native protein, but rather includes conformationally constrained structures that regenerate (exhibit) essential properties (such as binding properties) of such native structures.

The term “binding partner” or “binding partners” is used herein in the broadest sense and refers to two or more polypeptide sequences that are able to join each other, by covalent linkage or by non-covalent association, under in vitro and/or in vivo conditions. Examples of such binding partners include, without limitation, antibody and antigen, ligand and receptor, enzyme and substrate, liganded antibodies and anti-immunoglobulin antibodies recognizing such liganded antibodies, anti-idiotype antibodies and antibodies to which they bind, which may be isolated or be part of cells, tissues, organs or organisms in which they are naturally present or are introduced. Binding may take place by the association of more than two binding partners.

By “binding partner complex” or “target-binding partner complex” is meant the association of two or more binding partners (as hereinabove defined), such as a target and molecule binding to the target, which join each other in a specific, detectable manner; thus, for example, the association of ligand and receptor, antibody and antigen, enzyme and substrate, antibody and anti-idiotype antibody, liganded antibody and antibody binding thereto.

The term “solid support” is used herein to refer to an insoluble matrix to which a target and its candidate binding partners and target-binding partner complexes may be linked. The solid support is typically biological in nature, such as, without limitation, a cell, a spore, or a viral or a bacteriophage particle.

The terms “conjugate,” “conjugated,” and “conjugation” refer to any and all forms of covalent or non-covalent linkage, and include, without limitation, direct genetic or chemical fusion, coupling through a linker or a cross-linking agent, and non-covalent associate, for example using a leucine zipper.

The terms “tandem or multimeric assemblies,” “tandem assemblies,” and “multimeric assemblies” are used in the broadest sense and refer to two or more polypeptide fragments associated with each other by any means, including conjugation (as hereinabove defined) or by complexing, wherein the “fragments” may be identical to portions of native polypeptides and/or may be artificial sequences not present in a native polypeptide target. “Tandem assemblies” refer to the association of two fragments, while “multimeric assemblies” to the association of more than two fragments.

The term “fusion” is used herein to refer to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini.

As used herein, the terms “peptide,” “polypeptide” and “protein” all refer to a primary sequence of amino acids that are joined by covalent “peptide linkages.” In general, a peptide consists of a few amino acids, typically from about 2 to about 50 amino acids, and is shorter than a protein. The term “polypeptide,” as defined herein, encompasses peptides and proteins.

In the context of the present invention, the term “antibody” (Ab) is used in the broadest sense and includes polypeptides which exhibit binding specificity to a specific antigen as well as immunoglobulins and other antibody-like molecules which lack antigen specificity. Polypeptides of the latter kind are, for example, produced at low levels by the lymph system and, at increased levels, by myelomas. In the present application, the term “antibody” specifically covers, without limitation, monoclonal antibodies, polyclonal antibodies, and antibody fragments.

“Native antibodies” are usually heterotetrameric glycoproteins of about 150,000 daltons, composed of two identical light (L) chains and two identical heavy (H) chains. Each light chain is linked to a heavy chain by covalent disulfide bond(s), while the number of disulfide linkages varies between the heavy chains of different immunoglobulin isotypes. Each heavy and light chain also has regularly spaced intrachain disulfide bridges. Each heavy chain has, at one end, a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains, Chothia et al., J. Mol. Biol. 186:651 (1985); Novotny and Haber, Proc. Natl. Acad. Sci. U.S.A. 82:4592 (1985).

The term “variable” with reference to antibody chains is used to refer to portions of the antibody chains which differ extensively in sequence among antibodies and participate in the binding and specificity of each particular antibody for its particular antigen. Such variability is concentrated in three segments called hypervariable regions both in the light chain and the heavy chain variable domains. The more highly conserved portions of variable domains are called the framework region (FR). The variable domains of native heavy and light chains each comprise four FRs (FR1, FR2, FR3 and FR4, respectively), largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669). The constant domains are not involved directly in binding an antibody to an antigen, but exhibit various effector functions, such as participation of the antibody in antibody-dependent cellular toxicity.

The term “hypervariable region” when used herein refers to the amino acid residues of an antibody which are responsible for antigen-binding. The hypervariable region comprises amino acid residues from a “complementarity determining region” or “CDR” (i.e., residues 30-36 (L1), 46-55 (L2) and 86-96 (L3) in the light chain variable domain and 30-35 (H1), 47-58 (H2) and 93-101 (H3) in the heavy chain variable domain; MacCallum et al, J Mol Biol. 1996.

The term “framework region” refers to the art recognized portions of an antibody variable region that exist between the more divergent CDR regions. Such framework regions are typically referred to as frameworks 1 through 4 (FR1, FR2, FR3, and FR4) and provide a scaffold for holding, in three-dimensional space, the three CDRs found in a heavy or light chain antibody variable region, such that the CDRs can form an antigen-binding surface.

Depending on the amino acid sequence of the constant domain of their heavy chains, antibodies can be assigned to different classes. There are five major classes of antibodies IgA, IgD, IgE, IgG, and IgM, and several of these may be further divided into subclasses (isotypes), e.g., IgG1, IgG2, IgG3, IgG4, IgA, and IgA2.

The heavy-chain constant domains that correspond to the different-classes of immunoglobulins are called α, δ, ε, γ, and μ, respectively.

The “light chains” of antibodies from any vertebrate species can be assigned to one of two clearly distinct types, called kappa (κ) and lambda (λ), based on the amino acid sequences of their constant domains.

“Antibody fragments” comprise a portion of a full length antibody, generally the antigen binding or a variable domain thereof. Examples of antibody fragments include, but are not limited to, Fab, Fab′, F(ab′)₂, scFv, (scFv)₂, dAb, and complementarity determining region (CDR) fragments, linear antibodies, single-chain antibody molecules, minibodies, diabodies, multispecific antibodies formed from antibody fragments, and, in general, polypeptides that contain at least a portion of an immunoglobulin that is sufficient to confer specific antigen binding to the polypeptide.

The term “monoclonal antibody” is used to refer to an antibody molecule synthesized by a single clone of B cells. The modifier “monoclonal” indicates the character of the antibody as being obtained from a substantially homogeneous population of antibodies, and is not to be construed as requiring production of the antibody by any particular method. Thus, monoclonal antibodies may be made by the hybridoma method first described by Kohler and Milstein, Nature 256:495 (1975); Eur. J. Immunol. 6:511 (1976), by recombinant DNA techniques, or may also be isolated from phage or other antibody libraries.

The term “polyclonal antibody” is used to refer to a population of antibody molecules synthesized by a population of B cells.

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Plückthun in The Pharmacology of Monoclonal Antibodies, vol. 113, Rosenburg and Moore eds. Springer-Verlag, New York, pp. 269-315 (1994). Single-chain antibodies are disclosed, for example in WO 88/06630 and WO 92/01047.

Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., Proc. Natl. Acad. Sci. USA 90:6444 6448 (1993), and Poljak, R. J., et al., Structure 2:1121 1123 (1994)).

The term “minibody” is used to refer to an scFv-CH3 fusion protein that self-assembles into a bivalent dimer of 80 kDa (scFv-CH3)₂.

The term “aptamer” is used herein to refer to synthetic nucleic acid ligands that bind to protein targets with high specificity and affinity. Aptamers are known as potent inhibitors of protein function.

The term “affibody” is used to refer to engineered, target-specific, non-immunoglobulin binding proteins, which are typically based on the three-helix scaffold of the Z domain derived from staphylococcal protein A. The 58-amino acid Z domain is derived from one of five homologous domains (the B domain) in Staphylococcus aureus protein A (SPA). SPA binds strongly to the Fc region of immunoglobulins, and Z was originally developed as a stabilized gene fusion partner for affinity purification of recombinant proteins by using IgG-containing resins. The structure of a complex between the B domain of SPA and an Fc fragment shows that the binding surface consists of residues that are exposed on helices 1 and 2, whereas helix 3 is not directly involved in binding. Affibodies are usually selected from combinatorial libraries in which typically 13 residues at the Fc-binding surface of helices 1 and 2 are randomized. Specific binders to target proteins are then identified by biopanning the phage-displayed library against desired targets. Such affibodies can be used as an alternative to immunoglobulins in various biochemical assays and clinical applications.

A dAb fragment (Ward et al., Nature 341:544 546 (1989)) consists of a V_{H} domain.

One or more CDRs may be incorporated into a molecule either covalently or noncovalently to make it an “immunoadhesin.” An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest.

As used herein the term “antibody binding regions” refers to one or more portions of an immunoglobulin or antibody variable region capable of binding an antigen(s). Typically, the antibody binding region is, for example, an antibody light chain (VL) (or variable region thereof), an antibody heavy chain (VH) (or variable region thereof), a heavy chain Fd region, a combined antibody light and heavy chain (or variable region thereof) such as a Fab, F(ab′)₂, single domain, or single chain antibody (scFv), or a full length antibody, for example, an IgG (e.g., an IgG1, IgG2, IgG3, or IgG4 subtype), IgA1, IgA2, IgD, IgE, or IgM antibody.

The term “amino acid” or “amino acid residue” typically refers to an amino acid having its art recognized definition such as an amino acid selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val) although modified, synthetic, or rare amino acids may be used as desired. Thus, modified and unusual amino acids listed in 37 CFR 1.822(b)(4) are specifically included within this definition and expressly incorporated herein by reference. Amino acids can be subdivided into various sub-groups. Thus, amino acids can be grouped as having a nonpolar side chain (e.g., Ala, Cys, Ile, Leu, Met, Phe, Pro, Val); a negatively charged side chain (e.g., Asp, Glu); a positively charged side chain (e.g., Arg, His, Lys); or an uncharged polar side chain (e.g., Asn, Cys, Gln, Gly, His, Met, Phe, Ser, Thr, Trp, and Tyr). Amino acids can also be grouped as small amino acids (Gly, Ala), nucleophilic amino acids (Ser, His, Thr, Cys), hydrophobic amino acids (Val, Leu, Ile, Met, Pro), aromatic amino acids (Phe, Tyr, Trp, Asp, Glu), amides (Asp, Glu), and basic amino acids (Lys, Arg).

The term “polynucleotide(s)” refers to nucleic acids such as DNA molecules and RNA molecules and analogs thereof (e.g., DNA or RNA generated using nucleotide analogs or using nucleic acid chemistry). As desired, the polynucleotides may be made synthetically, e.g., using art-recognized nucleic acid chemistry or enzymatically using, e.g., a polymerase, and, if desired, be modified. Typical modifications include methylation, biotinylation, and other art-known modifications. In addition, the nucleic acid molecule can be single-stranded or double-stranded and, where desired, linked to a detectable moiety.

The term “mutagenesis” refers to, unless otherwise specified, any art recognized technique for altering a polynucleotide or polypeptide sequence. Preferred types of mutagenesis include error prone PCR mutagenesis, saturation mutagenesis, or other site directed mutagenesis. The term “vector” is used to refer to a rDNA molecule capable of autonomous replication in a cell and to which a DNA segment, e.g., gene or polynucleotide, can be operatively linked so as to bring about replication of the attached segment. Vectors capable of directing the expression of genes encoding for one or more polypeptides are referred to herein as “expression vectors.” The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Percent amino acid sequence identity may be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

The term “leucine zipper” is used to refer to a repetitive heptad motif typically containing four to five leucine residues present as a conserved domain in several proteins. Leucine zippers fold as short, parallel coiled coils, and are believed to be responsible for oligomerization of the proteins of which they form a domain.

The term “microarray” refers to an ordered arrangement of hybridizable array elements, such as polynucleotide probes, on a substrate.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

B. Detailed Description

Techniques for performing the methods of the present invention are well known in the art and described in standard laboratory textbooks, including, for example, Ausubel et al., Current Protocols of Molecular Biology, John Wiley and Sons (1997); Molecular Cloning: A Laboratory Manual, Third Edition, J. Sambrook and D. W. Russell, eds., Cold Spring Harbor, N.Y., USA, Cold Spring Harbor Laboratory Press, 2001; O'Brian et al., Analytical Chemistry of Bacillus Thuringiensis, Hickle and Fitch, eds., Am. Chem. Soc., 1990; Bacillus thuringiensis: biology, ecology and safety, T. R. Glare and M. O'Callaghan, eds., John Wiley, 2000; Antibody Phage Display, Methods and Protocols, Humana Press, 2001; and Antibodies, G. Subramanian, ed., Kluwer Academic, 2004. Mutagenesis can, for example, be performed using site-directed mutagenesis (Kunkel et al., Proc. Natl. Acad. Sci USA 82:488-492 (1985)). PCR amplification methods are described in U.S. Pat. Nos. 4,683,192, 4,683,202, 4,800,159, and 4,965,188, and in several textbooks including “PCR Technology: Principles and Applications for DNA Amplification”, H. Erlich, ed., Stockton Press, New York (1989); and PCR Protocols: A Guide to Methods and Applications, Innis et al., eds., Academic Press, San Diego, Calif. (1990).

The present invention concerns physically selectable displays of conformationally constrained polypeptide targets, such as conformational epitope libraries. According to the present invention tandem or multimeric assemblies of discrete or random fragments of native polypeptides, or sequences mimicking such fragments, are displayed in a selectable manner and screened with a library of candidate binding partners. Due to their proximity, and/or due to the presence of a structural support element, the fragments present in the tandem or multimeric assemblies, that are part of a three-dimensional structural element, such as a conformational epitope, will assume the proper conformationally constrained three-dimensional structure and recreate the essential properties of the structural element in question, such as a conformational epitope: The conformationally constrained structures can then be screened and identified by using candidate binding partners. Thus, conformational epitopes can be identified by screening a selectable display of tandem or multimeric assemblies of polypeptide fragments with an antibody library, and selecting the matching target-antibody collection. This way, antibodies can be generated to all possible conformational epitopes of all proteins. In a more generic aspect, this approach is suitable for parallel selection of conformationally constrained polypeptide structures and binding partners, such as antibodies, scaffolds, proteins, peptides, and the like.

Thus, the present invention includes cloning of tandem or multimeric assemblies of expressable gene fragments, and screening the collection of the assemblies cloned against a library of candidate binding partners, such as an antibody library. The matching target and binding partner (e.g. antibody) collection is then enriched and segregated, and the target and binding (e.g. antibody) sequences can be independently recovered and sequenced.

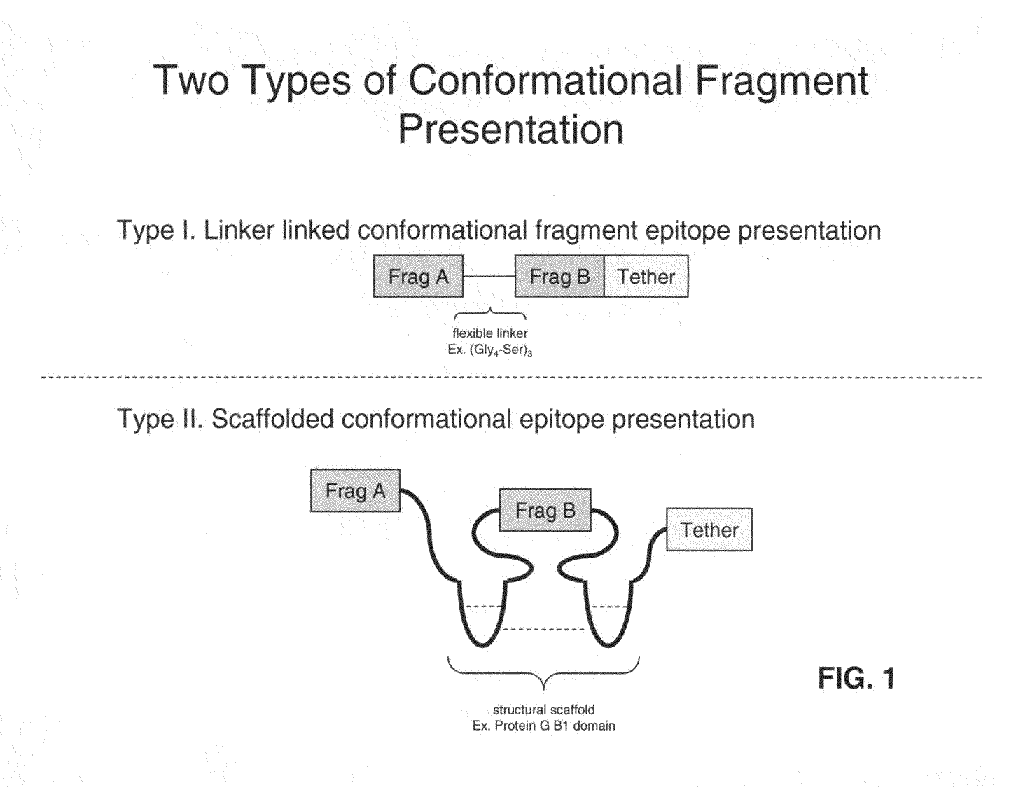

In the first step, two or more cDNA fragments are cloned, using standard cloning schemes, sequentially into two or more cloning sites of an expression vector. The sites may be separated by a synthetic linker, present on a scaffold capable of presenting a free amino or carboxy terminus and a constrained loop, or, in some instances, may be directly fused to each other. Two representative examples of conformational fragment presentation are illustrated in FIG. 1, and described in Example 1.

In a particular embodiment, the fragments participating in the tandem or multimeric assemblies are directly fused to each other, and are produced by expression of the nucleic acid encoding the fused polypeptide sequences. Alternatively, the coding sequences may be linked by extraneous expressable sequences that result in the expression of the polypeptide in which the tandem or multimeric fragments are connected by an extraneous sequence. Expressable sequences include peptide linkers that are of sufficient length to provide flexibility and allow movement of the fragments connected so that they can assume the desired conformation, but are short enough the keep the fragments connected in close proximity, so that the conformational change can be induced. Such linkers are usually between about 3 to about 25 residues, or about 5 to about 20 residues, or about 8 to about 15 residues, or about 10 to about 15 residues in length, although longer linkers may also be used, depending on the nature of the fragments connected.

In another embodiment, at least one fragment participating in a tandem or multimeric assembly is structurally constrained, and thus can, for example, be a helical or β-sheet structure, or a motif characteristics of or more protein families, such as a helical bundle, a trifoil structure, a membrane-spanning helix, or an extracellular loop. Thus, it is possible to present single linear or discontinuous sequences of a target protein on a surrogate scaffold, such that the introduced sequence adopts partial or total conformational elements of the original protein. An example of this approach is described in Example 3.

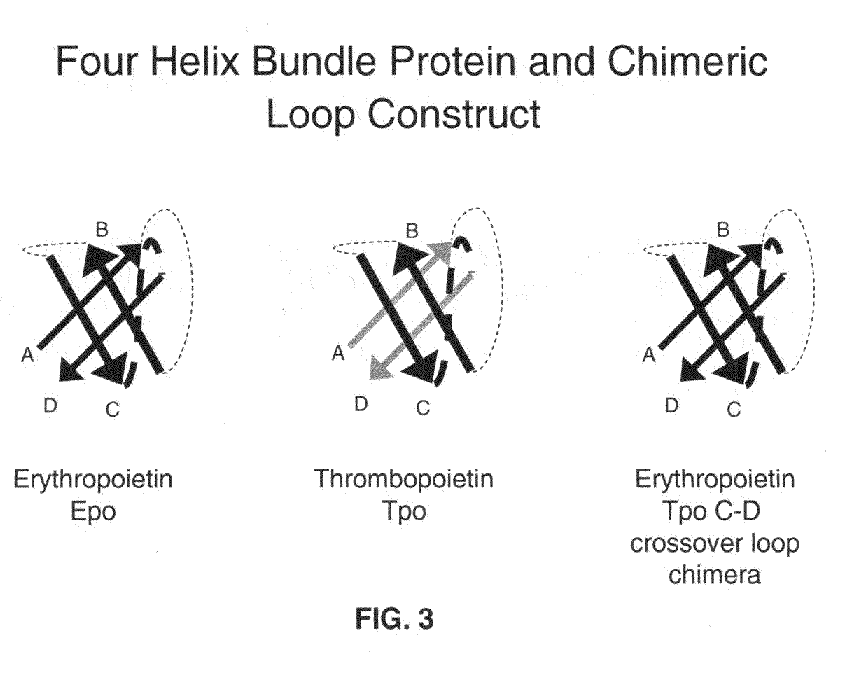

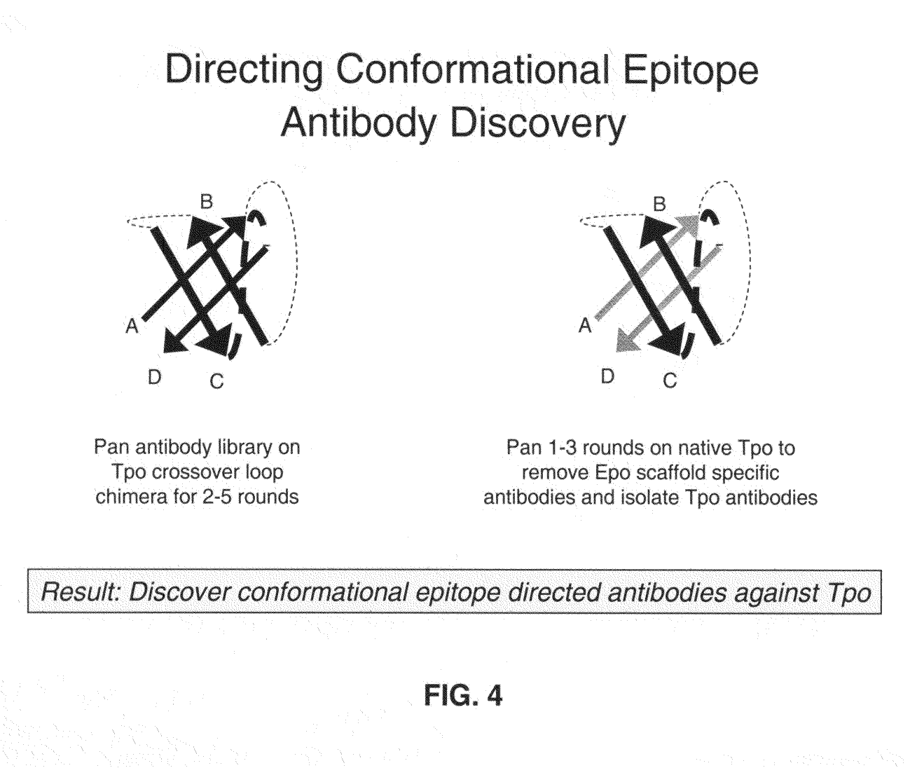

In addition to thrombopoietin (TPO) and erythropoietin (EPO) illustrated in Example 3 and shown in FIGS. 3 and 4, a four-helix structure, containing four antiparallel helical bundles, is a common structural scaffold for many cytokines, such an interleukins, e.g. IL-1, IL-2, IL-3, IL-4, IL-5, IL-6, IL-10, IL-11, IL-12, IL-15, IL-17, IL-18, IL-23, and their respective family members, colony stimulating factor (CSF), granulocyte colony stimulating factor (G-CSF), and granulocyte macrophage colony stimulating factor (GMCSF), as well as several growth factors. A reference four-helix bundle from one cytokine can be used as a structural scaffold for presentation of epitopes from other helical bundle proteins, such as other cytokines. Similarly, extracellular loops from receptors, such as seven-transmembrane receptors, can be used as scaffold to display epitopes from the same or different proteins. This approach enables facile and directed antibody selection of antibodies against all known four helix bundle proteins, or proteins characterized by the presence of another well-defined structural motif. Alternatively, these scaffolds may be engineered to contain multiple loops, single or multiple helical substitutions, or even combinations of loops and helices from any number of target proteins. By extension, other conserved structural elements of soluble and single span proteins can be utilized to present and direct antibody recognition to their critical elements within cognate superfamily proteins. Thus, portions of the extracellular domains of multispan G-protein coupled receptors can be grafted to an unrelated protein scaffold, as illustrated in Example 4.

Other scaffolds that can be used in the methods of the present invention are described in Binz et al., Nature Biotechnology 23(10):1257-1268 (2005), the entire content of which is hereby expressly incorporated by reference. Such scaffolds include, without limitation, CTLA-4, tandamistat, fibronectin, neocarzinostatin, CMB4-2, lipocalins, T-cell receptor, protein A domain Protein Z), 1m9, designed AR proteins, zinc finger, pVIII, avian pancreatic polypeptide, GCN4, WW domain, Src homology domain 3 (SH3), Src homology domain 2 (SH2), PDZ domains, TEM-1 β-lactamase, GFP, thioredoxin, staphylococcal nuclease, PHD-finger, Cl-2, BPTI, APPI, HPSTI, ecotin, LACI-D1, LDT-I, MTI-II, scorpion toxins, insect defensin A peptide, EETI-II, Min-23, CBD, PBP, cytochrome B₅₆₂, Ld1 receptor domain A, γ-crystallin, ubiquitin, transferrin, C-type lectin-like domain, Avimers (Avidia/Amgen) and microproteins (Amunix).

The tandem or multimeric fragments can be derived from any known source of polynucleotides, including single genes, differentiated cells, tissues, organs or organisms. Thus, for example, coexpression of a random or desired fragment from a particular gene with other fragments of the same gene and subsequent screening with an antibody library will yield antibodies to the desired epitope as well as antibodies to all epitopes of the single target. This approach also provides a strategy to convert expressed genes to a physically represented clonal collection for antibody selection, obviating the need for cloning full-length genes.

In another embodiment, results of a microarray or gene amplification study can be analyzed for differential expression (over- or under-expression) of genes and their structural determinants. In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106-149 (1996)). Microarray analysis can be performed by commercially available equipment, following manufacturer's protocols, such as by using the Affymetrix GenChip technology, or Incyte's microarray technology.

The development of microarray methods for large-scale analysis of gene expression makes it possible to search systematically for molecular markers of cancer classification and outcome prediction in a variety of tumor types.

After identifying the differentially expressed determinants (such as determinants over- or under-expressed in a diseased tissue, e.g. a cancer tissue, relative to a normal tissue of the same cell type), they can be re-synthesized, for example by known chemical methods of peptide synthesis. This re-synthesis process is expected to normalize these determinants and yield a directed physical library with a vast depth of combinatorial clonable components.

Removal of RNA or total DNA from target cells, and subsequent screening of tandem or multimeric assembly of expressed sequences from such RNA or DNA in accordance with the present invention enables the identification of all epitopes from target tissues, organs or organisms. Thus, for example, this embodiment allows the identification of antibodies to antigenic determinants from stem cells, diseased tissues, activated immune cells, etc. General methods for RNA and DNA extraction are well known in the art and are disclosed in standard textbooks of molecular biology, including Ausubel et al., Current Protocols of Molecular Biology, John Wiley and Sons (1997). Methods for RNA extraction from paraffin embedded tissues, such as cancer biopsy samples, are disclosed, for example, in Rupp and Locker, Lab Invest. 56:A67 (1987), and De Andrés et al., BioTechniques 18:42044 (1995). In particular, RNA isolation can be performed using a purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from a tumor can be isolated, for example, by cesium chloride density gradient centrifugation.

Cloning and expression vectors are well known in the art and are commercially available. The vector components generally include, but are not limited to, one or more of the following: a signal sequence, an origin of replication, one or more marker genes, an enhancer element, a promoter, and a transcription termination sequence.

Suitable host cells for cloning or expressing the DNA in the vectors herein are prokaryote, yeast, or higher eukaryote (mammalian) cells. Suitable prokaryotes include Gram-negative or Gram-positive organisms, for example, Escherichia, e.g., E. coli, Enterobacter, Erwinia, Klebsiella, Proteus, Salmonella, e.g., Salmonella typhimurium, Serrafia, e.g, Serratia marcescans, and Shigeila, as well as Bacilli such as B. subtilis, B thuringiensis and B. licheniformis (e.g., B. licheniformis 41P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X 1776 (ATCC 31,537), and E. coli W3110 (ATCC 27,325) are suitable. These examples are illustrative rather than limiting.

Suitable yeasts include Saccharomyces cerevisiae, or common baker's yeast. In addition, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Examples of invertebrate multicellular organisms include plant and insect cells, including insect host cells from hosts such as Spodoptera frugiperda (caterpillar), Aedes aegypti (mosquito), Aedes albopictus (mosquito), Drosophila melanogaster (fruitfly), and Bombyx mori. Viral strains for transfection of insect cells include, for example, the L-1 variant of Autographa californica NPV and the Bm-5 strain of Bombyx mori NPV. Plant cell cultures of cotton, corn, potato, soybean, petunia, tomato, and tobacco can also be utilized as hosts.

Examples of suitable mammalian host cell lines include, without limitation, monkey kidney CV1 line transformed by SV40 (COS-7, ATCC CRL 1651); human embryonic kidney line 293 (293 cells) subcloned for growth in suspension culture, Graham et al, J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

The host cells may be cultured in a variety of media. Commercially available media include Ham's F10 (Sigma), Minimal Essential Medium ((MEM), (Sigma), RPMI-1640 (Sigma), and Dulbecco's Modified Eagle's Medium ((DMEM), Sigma). In addition, any of the media described in Ham et al., Meth. Enz. 58:44 (1979) and Barnes et al., Anal. Biochem. 102:255 (1980) may be used as culture media for the host cells. The culture conditions, such as temperature, pH, and the like, are those previously used with the host cell selected for expression, and are included in the manufacturer's instructions or will otherwise be apparent to the ordinarily skilled artisan

In one embodiment, both/all fragments making up a tandem or multimeric assembly are randomly selected. This approach may, for example, be used to express all expressable epitopes from a targeted population.

As discussed earlier, systems for displaying heterologous proteins, including antibodies and other polypeptides, are well known in the art. Antibody fragments have been displayed on the surface of filamentous phage that encode the antibody genes (Hoogenboom and Winter J. Mol. Biol., 222:381 388 (1992); McCafferty et al., Nature 348(6301):552 554 (1990); Griffiths et al. EMBO J., 13(14):3245-3260 (1994)). For a review of techniques for selecting and screening antibody libraries see, e.g., Hoogenboom, Nature Biotechnol. 23(9):1105-1116 (2005). In addition, there are systems known in the art for display of heterologous proteins and fragments thereof on the surface of Escherichia coli (Agterberg et al., Gene 88:37-45 (1990); Charbit et al., Gene 70:181-189 (1988); Francisco et al., Proc. Natl. Acad. Sci. USA 89:2713-2717 (1992)), and yeast, such as Saccharomyces cerevisiae (Boder and Wittrup, Nat. Biotechnol. 15:553-557 (1997); Kieke et al., Protein Eng. 10:1303-1310 (1997)). Other known display techniques include ribosome or mRNA display (Mattheakis et al., Proc. Natl. Acad. Sci. USA 91:9022-9026 (1994); Hanes and Pluckthun, Proc. Natl. Acad. Sci. USA 94:4937-4942 (1997)), DNA display (Yonezawa et al., Nucl. Acid Res. 31(19):e118 (2003)); microbial cell display, such as bacterial display (Georgiou et al., Nature Biotech. 15:29-34 (1997)), display on mammalian cells, spore display (Isticato et al., J. Bacteriol. 183:6294-6301 (2001); Cheng et al., Appl. Environ. Microbiol. 71:3337-3341 (2005) and co-pending provisional application Ser. No. 60/865,574, filed Nov. 13, 2006), viral display, such as retroviral display (Urban et al., Nucleic Acids Res. 33:e35 (2005), display based on protein-DNA linkage (Odegrip et al., Proc. Acad. Natl. Sci. USA 101:2806-2810 (2004); Reiersen et al., Nucleic Acids Res. 33:e10 (2005)), and microbead display (Sepp et al., FEBS Lett. 532:455-458 (2002)).

For the purpose of the present invention, the tandem or multimeric assemblies of polypeptide fragments (e.g. tandem and/or multimer antigen fragments) may be advantageously displayed using spore display, including surface display system using a component of the Bacillus subtilis spore coat (CorB) and Bacillus thuringiensis (Bt) spore display, as described in Isticato et al., J. Bacteriol. 183:6294-6301 (2001); Cheng et al., Appl. Environ. Microbiol. 71:3337-3341 (2005), and co-pending provisional application Ser. No. 60/865,574, filed Nov. 13, 2006, the entire disclosures of which is hereby expressly incorporated by reference.

Spore display systems are based on attaching the sequences to be displayed to a coat protein (such as a Bacillus subtilis spore coat protein) or to a toxin-protoxin (such as a Bt protoxin sequence). An advantage of spore display systems is the homogenous particle surface and particle size of non-eukaryotic nature, which is expected to provide an ideal non-reactive background. In addition, the particle size of spores is sufficient to enable selection by flow cytometry that permits selectable clonal isolation, based upon interactions.

Leveraging on the stability of spores, it is possible to perform various post-sporulation chemical, enzymatic and/or environmental treatments and modification. Thus, it is possible to stabilize structural helical structures with chemical treatment using trifluoroethanol (TFE), when such structures are displayed. In addition, oxidative stress treatments, such as treatments with Reactive Oxygen Species (e.g. peroxide) or reactive Nitrogen Species (e.g. nitrous acid) are possible. It is also possible to expose defined or crude populations of spore-displayed polypeptides to enzymatic treatments, such as proteolytic exposure, other enzymatic processes, phosphorylation, etc. Other possible treatments include, without limitation, nitrosylation by peroxynitrite treatment, proteolysis by recombinant, purified, or serum protease treatment, irradiation, coincubation with known chaperones, such as heat shock proteins (both bacterial and mammalian), treatment with folding proteins, such as protein disulfide isomerase, prolyl isomerase, etc., lyophilization, and preservative-like treatments, such as treatment with thimerosol. These treatments can be performed by methods well known in the art.

Finally, phage-displayed antibody clones can be co-captured to wells with individual spores bearing cognate antigens. This enables multiplexed co-segregation and rescue of antigen-antibody pairs. This can be similarly extended to the selection and rescue of other binding partners as well.

In brief, in the Bt spore display system, Bt protoxin sequences can be obtained from native Bt protoxin proteins, produced by chemical synthesis or methods of recombinant DNA technology, or by any other technique known in the art. Native Bt protoxin proteins or their coding sequences can be isolated from various Bt subspecies, such as, for example, subspecies kurstaki, dendrolimus, galleriae, entomocidus, aizawai, morrisoni, tolworthi, alesti, or israelensis.

Thus, DNA encoding the Bt protoxin fragments can be PCR amplified from the chromosome of a suitable Bt subspecies using appropriate oligonucleotide primers and probes, by methods known in the art. The PCR product can then be purified by known techniques, such as, for example, by using the QIAquick gel extraction kit (Qiagen) following the manufacturer's instructions.

Recombinant host cells suitable for cloning the protoxin fragments include prokaryote, yeast, or higher eukaryote cells. For cloning and routine plasmid manipulation the preferred host is E. coli.

The Bt protoxin sequences are then used to display the tandem or multimeric assemblies of the present invention on the surface of Bt spores. Thus, the present invention also concerns conjugates of Bt protoxin sequences and such tandem or multimeric assemblies of polypeptide sequences.

Conjugation of the tandem or multimeric assemblies of the present invention to Bt protoxin sequences may be performed by fusion, preferably at a terminal end, such as the N- or C-terminus of the Bt protoxin sequence. Alternatively, an appropriate peptide linker sequence can be used to prepare the conjugates.

The linker sequence separates the displayed assembly and the Bt protoxin sequence by a distance sufficient to ensure that each sequence can assume a proper conformation (conformationally constrained structure), if the fragments present in the sequence are capable of forming such structure. The length of the linker sequence may vary and generally is between 1 and about 50 amino acids, more commonly, up to about 15 amino acids, or up to about 10 amino acids, or up to about 8 amino acids, or up to about 7 amino acids, or up to about 5 amino acids, or up to about 3 amino acids long. The linker sequence is incorporated into the conjugate by methods well known in the art.

In order to facilitate removal of the displayed molecule, the linker may include a sequence that is a substrate for an enzyme, such as a protease. Thus, in a specific embodiment, the natural substrate of a given protease can be used as or included in the linker peptide. For instance, the linker can be, or can include, the substrate site of the tobacco etch virus (TEV) (ENLYFOG). Alternatively, the linker peptide may be different from the natural substrate of a protease, but may include sequences that can be cleaved by the protease. Thus, it is known that trypsin-like proteases specifically cleave at the carboxyl side of lysine and arginine residues, while chymotrypsin-like proteases are specific for cleavage at tyrosine, phenylalanine and tryptophan residues, etc.

The linkage between the protoxin sequence and the tandem or multimeric assembly to be displayed, can be achieved by using a heterodimeric motif, where the two components forming the dimer can be binding partners which are covalently associated with each other, or may associate through non-covalent interaction.

Covalent association may, for example, take place through the formation of a disulfide bond between cysteines of the binding partners. The disulfide bond can be broken and the displayed assembly released by treatment with a reducing agent that disrupts the disulfide bond, such as, for example, dithiothreitol, dithioerythritol, β-mercaptoethanol, phosphines, sodium borohydride, and the like. Preferably, thiol-group containing reducing agents are used.

Non-covalent association can be achieved, for example, using a pair of leucine zipper peptides. Leucine zippers were originally identified in several DNA-binding proteins (Landschulz et al., Science 240: 1759, 1988). Thus, the leucine zipper domain is a term used to refer to a conserved peptide domain present in these and proteins, which is responsible for dimerization of the proteins. The leucine zipper domain comprises a repetitive heptad repeat, typically with four or five leucine residues interspersed with other amino acids.

Leucine zipper peptides include, for example, the well known

c-Jun “leucine zipper peptide”

(SEQ ID NO: 1)

RIARLEEKVKTLKAQNSELASTANMLREQVAQLKQKVMNY
and the
v-Fos “leucine zipper peptide”

(SEQ ID NO: 2)

LTDTLQAETDQLEDKKSALQTEIANLLKEKEKLEFILAAY

The products of the nuclear oncogenes fos and jun comprise leucine zipper domains which preferentially form a heterodimer (O'Shea et al., Science 245:646, 1989; Turner and Tjian, Science 243:1689, 1989).

Other examples of leucine zipper peptides include, without limitation, domains found in the yeast transcription factor GCN4 and a heat-stable DNA-binding protein found in rat liver (C/EBP; Landschulz et al., Science 243:1681, 1989); the gene product of the murine proto-oncogene, c-myc (Landschulz et al., Science 240:1759, 1988). The fusogenic proteins of several different viruses, including paramyxovirus, coronavirus, measles virus and many retroviruses, also possess leucine zipper domains (Buckland and Wild, Nature 338:547,1989; Britton, Nature 353:394, 1991; Delwat and Mosialos, AIDS Research and Human Retroviruses 6:703, 1990). It is often preferred to use synthetic, as opposed to naturally occurring, leucine zipper peptides, since the synthetic sequences can be designed to exhibit improved properties, such as stability.

In order to produce the fusions of the present invention, the amplified Bt protoxin DNA fragment can be cloned into an appropriate plasmid in frame with the coding sequence of the assembly of the tandem or multimeric assemblies to be displayed, under control of a suitable sporulation-specific promoter. The sporulation specific promoter can, but does not have to be, obtained from the same Bt subspecies from which the Bt protoxin fragment originates.

The plasmids containing the coding sequences for the protoxin fragment—heterologous polypeptide fusions can be introduced into Bt by electroporation, essentially as described by Du et al., Appl. Environ. Microbiol. 71(6):3337-3341 (2005), following the method of Macaluso and Mettus, J. Bacteriol. 173:1353-1356 (1991).

After plasmid transformation of the target Bt strain, the cells are grown in an appropriate medium to promote sporulation. As a result, the Bt spores will display on their surfaces the heterologous peptide or polypeptide present in the fusion.

The toxin-displayed molecule conjugates are referred to as being attached to the spore surface, however, in fact the toxin component reaches inside the spore coat, since the protoxins participating in the conjugates herein are part of the spore coat, although they are not coat proteins.

Similar techniques can be used in all spore display systems, including displays where the attachment is to a spore coat protein, including, for example, the spore display systems disclosed in U.S. Patent Application Publication Nos. 20020150594; 20030165538; 20040180348; 20040171065; and 20040254364.

The binding partners, such as antibodies, may be advantageously displayed using phage display. In phage display, the heterologous protein, such as a single-chain antibody fragment (scFv), is linked to a coat protein of a phage particle, while the DNA sequence from which it was expressed is packaged within the phage coat. Details of the phage display methods can be found, for example, McCafferty et al., Nature 348, 552-553 (1990)), describing the production of human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g. Johnson, Kevin S. and Chiswell, David J., Current Opinion in Structural Biology 3, 564-571 (1993). Several sources of V-gene segments can be used for phage display. Clackson et al., Nature 352 624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222, 581-597 (1991), or Griffith et al., EMBO J. 12, 725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al., Bio/Technol. 10, 779-783 (1992)). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the nM range. A strategy for making very large phage antibody repertoires has been described by Waterhouse et al., Nucl. Acids Res. 21, 2265-2266 (1993).

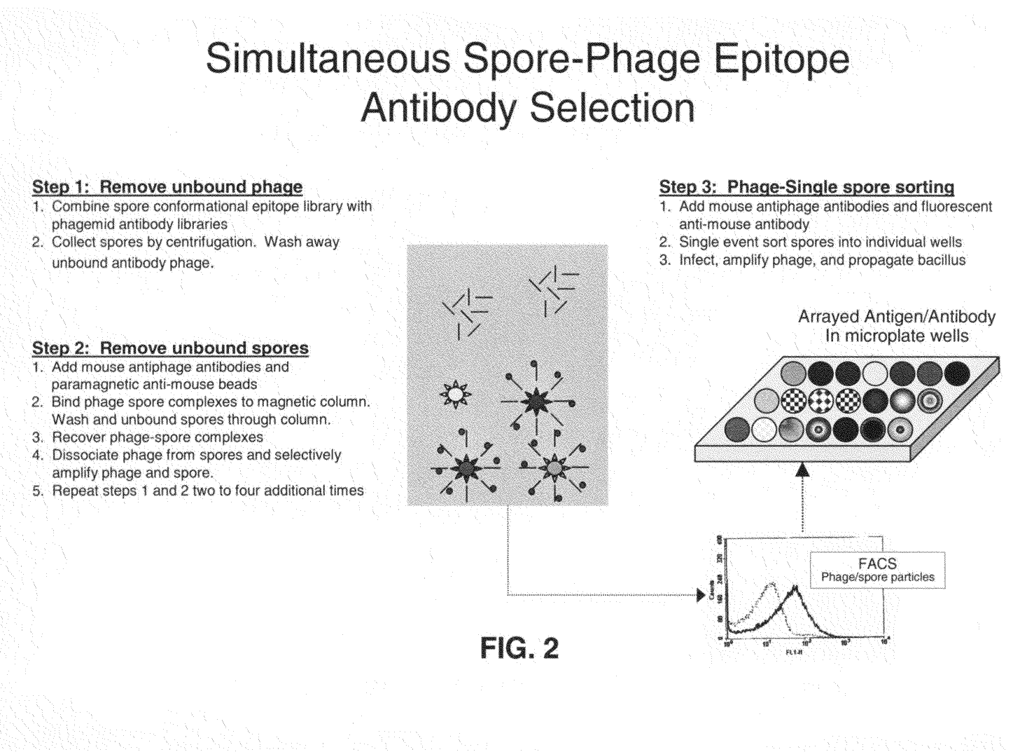

The simultaneous selection of an epitope library displayed on spores and a phage-display of an antibody library is illustrated in FIG. 2. In brief, in step 1, the spore-displayed conformational epitope library is combined with the phagemid antibody library. The spores are collected by centrifugation, and the unbound antibody phage is washed away. Next, the unbound spores are removed by adding mouse anti-phage antibodies and paramagnetic anti-mouse beads. The phage-spore complexes are bound to the magnetic column, which is then washed to remove the unbound spores. Following these steps, the phage-spore complexes can be recovered, the phage can be dissociated from the spores and the phage and spore can be selectively amplified. The foregoing steps can be repeated as needed, usually two to four additional times. In step 3, spores can be sorted into individual microplate wells, for example, by adding mouse anti-phage antibodies and detectably labeled (e.g. gluorescent) anti-mouse antibodies. The phage can then be amplified, and the bacillus (e.g. B. thuringiensis) carrying the spore-displayed sequences, propagated.

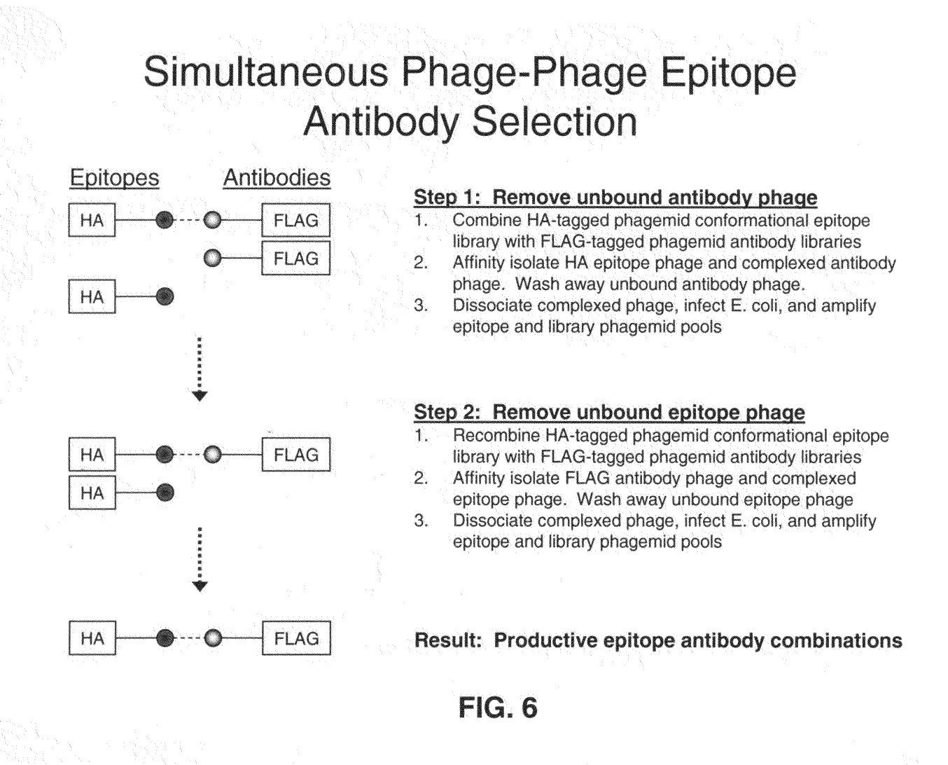

Simultaneous selection of a conformational epitope and antibody library is also possible, if each is displayed using the same carrier. An exemplary method for the simultaneous selection of conformational epitope and antibody libraries, each presented as a phage display, is shown in FIG. 6. In step 1, the unbound antibody phage is removed. First, the phagemid conformational epitope library labeled by an appropriate tag (e.g. HA) is combined with the phagemid antibody library labeled with a different tag (e.g. FLAG). The HA epitope phage and the complexed antibody phage are isolated by affinity purification, and the unbound antibody phage is washed away. Next, the complexed phage is dissociated, and the epitope and library phagemid pools are amplified, for example, using E. coli as a host. In step 2, the unbound epitope phage is removed. First, the HA-tagged phagemid conformational epitope library is recombined with the FLAG-tagged phagemid antibody library, and the FLAG antibody phage and complexed epitope phage are isolated by affinity purification. The unbound epitope phage is washed away, the complexed phage is dissociated and the epitope and library phagemid pools are amplified, for example, using E. coli host cells.

Although the invention has been illustrated with reference to certain phage and spore display systems, it is no so limited. The phage and spore display systems are merely illustrative, and other displays, whether specifically mentioned herein or not, are also suitable to practice the invention.

Further details of the invention are provided in the following non-limiting Examples.

### Example 1

### Tandem Assembled Conformational cDNA Fragments (FIGS. 1 and 2)

To assemble all expressable epitopes from a targeted population, first a collection of expressed genes from that targeted population is provided. In the present case, the goal is to capture all possible conformational epitopes from activated lymphocytes, but the method described herein is equally suitable for capturing conformational epitopes from any source.

First, peripheral blood mononuclear cells are isolated from three individuals by collecting whole blood into BD Vacutainer® CPT™ cell preparation tubes. Next, the lymphocytes are activated by mixing 1e7 cells from each of the three collections together followed by incubation for 6 hours. Following incubation, whole RNA is extracted by Tri reagent (Sigma) from fresh or RNA later stabilized tissue. Subsequently, the isolated donor total RNA is further purified to mRNA using oligotex purification (Qiagen). Next, first strand cDNA synthesis is generated by using random nonamer oligonucleotides and/or oligo (dT)₁₈ primers according to the protocol of AccuScript reverse transcriptase (Stratagene). Briefly, 100 ng mRNA, 0.5 mM dNTPs and 300 ng random nonamers and or 500 ng oligo (dT)₁₈ primers in Accuscript RT buffer(Stratagene) are incubated at 65° C. for 5 min, followed by rapid cooling to 4° C. Then, 10× reverse transcriptase reaction buffer (Stratagene), 100 mM DTT, Accuscript. RT, and RNAse Block are added and incubated 42° C. for 1 h, and the reverse transcriptase is inactivated by heating at 70° C. for 15 minutes. These cDNA fragments can then be cloned, using standard cloning schemes, sequentially into two tandem cloning sites of an expression vector. The tandem sites are either separated by a synthetic linker or present on a scaffold capable of presenting a free amino or carboxy terminus and a constrained loop. (FIG. 1, Type I and Type II). In the present case, these expressed tandem fragments are tethered to the surface of a spore in the form of recombinant fusions to a Bacillus thuringiensis protoxin.

The resulting spore-displayed collection is screened against a combinatorial antibody library to enrich for clones that are reactive against activated lymphocyte conformational epitopes. For screening, one milliliter containing 10-100 million Bacillus Thuringiensis spores are mixed with 10-100 billion phage and the collection is incubated for two hours at room temperature. Following this incubation, the spore collection and the antibodies bound are harvested by centrifugation. Centrifugation removes all unbound phage, resulting in a collection of both unbound spores and spore-phage complexes. Next the spore-phage complexes are positively selected by incubating the mixture with mouse monoclonal anti-phage antibodies and either anti-mouse paramagnetic nanoparticles (Miltenyi) for magnetic selection or fluorescently conjugated anti-mouse antibodies for FACS-based enrichment or isolation (FIG. 2). The complex is then treated with pH 2.2 glycine for ten minutes and then neutralized with 2M Tris. Following this acid elution, the antibody-antigen interaction is irreversibly disrupted and the phage can be amplified away from the spore with E. coli. and the bacillus re-propagated and selected. Alternatively, the phage and spores coding DNA could be rescued and amplified using other molecular biological techniques, such as PCR.

Following 3-5 rounds of selection, the antibody pools are expected to be sufficiently enriched to test or select further on activated lymphocytes. To demonstrate a specific enrichment, individuals, or pools of antibody clones, are prepared as soluble antibody fragments and tested for their ability to stain activated lymphocyte populations by flow cytometry.

Specifically, 200 ml cultures of antibody clones are grown in the E. coli strain HB2151 overnight at 30° C. in 2-YT supplemented with 50 μg/ml Ampicillin and 100 μM IPTG. Following this overnight growth, the cells are harvested by centrifugation. To isolate the accumulated antibody proteins in their periplasm, the cells are first re-suspended in 10 ml BBS-10E (200 mM Boric acid, 150 mM sodium chloride, and 10 mM EDTA). Next, 5 ml BBS-10EL (200 mM Boric acid, 150 mM sodium chloride, 10 mM EDTA, and 10 mg/ml lysozyme) are added and the mixture is place in an orbital shaker for 60-90 minutes at 37° C. Following this incubation, the cellular debris is removed by centrifugation from the antibody containing lysate. This lysate is next incubated with parental spores to detect binding to the cognate epitope. Specifically, spores are blocked with PBS containing 3% BSA, for 15 minutes at room temperature in a final volume of 0.1 ml. To these blocked spores, 0.1 ml lysate is added and the mixture is incubated for 1 hour at 4° C. Next, washing is performed by adding 0.8 ml PBS and then the spores are re-isolated by centrifugation. Then, a mouse monoclonal anti-His6 antibody is used to detect antibody binding to the spores by incubation for 30-60 minutes at 4° C. Following another wash, binding is detected with an appropriate anti-mouse phycoerythrin fluorophore conjugate.

The cognate antigens to any specific antibodies can be identified by traditional proteomic and molecular biological methods. Alternatively, the specifically identified antibody can be used to recover its cognate spore clone(s) containing the conformational epitope. As the epitope is encoded on a spore borne plasmid, it can be sequenced and used to identify the parental gene or genes from publicly available sequence databases.

The process described in the present example is expected to generate all possible antigens for any particular population of cells or tissue of interest. Because these fragments are random, not only conformational epitopes can assessed, but also discontinuous epitopes from single and even multiple proteins.

### Example 2

### Single Gene Conformational Fragments

In generating antibodies against a specified antigen, typically one finds a humoral response directed against an immunodominant epitope or epitopes. In many cases a desirable functional antibody may need to recognize a less immunodominant epitope. In this case one often tries to block the immunodominant epitope with an antibody and then reimmunizes or selects. The humoral response is then directed against the next most accessible epitope that still may or may not produce the desired antibody. As the goal of new antibody discovery is to generate an antibody against functional conformational epitopes, it follows that one would express such an epitope (or epitopes) for selection. A single epitope might only produce a response against a linear determinant where the resulting antibodies might not recognize the native protein. To increase the likelihood of generating a conformational antibody against such a native protein, this epitope needs to be expressed within the context of some critical structural element in order to approximate the native protein.

Accordingly, the desired epitope is synthetically assembled into a single site of a tandem construct and then the second site is supplemented with fragments from the parental protein. This second fragment serves as a structural conformational catalyst. In a particular embodiment, the first fragment is fixed with a single sequence and then random fragments from the parental protein are inserted into the second site, which is then recombinantly tethered to a bacillus carrier, such as a Bacillus Thuringiensis spore coat protein. Next, this collection is screened against a combinatorial antibody library, as described in Example 1. Following 3-5 rounds of double selection, this enriched antibody collection is counter selected against the native protein. The bound antibodies recognizing the native structure are retained, amplified, and identified. Conformational catalysis is expected to provide general structural support that may also be provided by a related structural surrogate. Therefore as another option for the second site, a single motif or collection of surrogate structural motifs is incorporated to complement and “catalyze” conformational epitope formation.

In this example, a forced recognition of a single epitope is described. However, the fragments can also be randomized such that expression of all possible epitopes is segregated. In this instance, all possible epitopes to a single protein can be simultaneously screened, thereby generating all possible antibody solutions to a particular protein

### Example 3

### Soluble Superfamily Conformational Antigens (FIGS. 3 and 4)

It is possible to present single linear or discontinuous sequences of a target protein on a surrogate scaffold, such that the introduced sequence adopts partial or total conformational elements of the original protein. In this example a known target-antibody interaction is recreated between Thrombopoietin (Tpo), a four-helix bundle cytokine, and a neutralizing antibody. The anti-Tpo antibody, TN1, recognizes the crossover loop from Tpo. When this loop is superimposed upon a closely related surrogate four helix bundle protein, such as Erythropoietin (Epo); it is expected to possess sufficient structural conformation such that the TN1 antibody binds the chimeric protein.

Specifically two types of Epo-Tpo loop proteins are constructed. The first substitutes amino acids 57 to 61 from Tpo for amino acids 57 to 61 in Epo. This corresponds to the crossover loop. As contextual presentation of the loop may be critical, another Epo-Tpo loop protein is also made, additionally containing the adjacent helix sequences from Tpo. In this case amino acids 53 to 68 from Tpo are substituted for amino acids 53 to 68 in Epo. The borders are defined by conserved sequences found in both Tpo and Epo with the intention they might provide even more conformational stability to the crossover loop. As the crystal structure of the TN1 antibody has shown some minor proximal contacts with the B helix we, both of the preceding Tpo loop constructs are also made in the background of Epo-Tpo mutants containing corresponding Tpo substitutions of amino acids 110 to 125 from Tpo for amino acids 114 to 139 in Epo as well as amino acids 97 to 134 from Tpo for amino acids 102 to 148 in Epo.

The Epo-Tpo loop proteins described above are recombinantly fused to the bacillus protoxin spore display system and the spores are screened against the TN1 antibody. These constructs are expected to bind and enrich the TN1 antibody selectively over an unrelated negative control antibody in a single round of panning. Next we would screen the best enriching ETL protein against a combinatorial antibody library through 3-5 rounds of spore panning. After this is completed the enriched antibodies are counter selected for binding to native Tpo protein. As a result, any anti-Tpo antibodies that are identified will be de facto binders of the Tpo crossover loop.

As numerous known cytokines and growth factors are four helix bundle proteins with highly variable loop structures, a surrogate four helix bundle scaffold, such as Epo, can be utilized to display a library of loops from known four helix bundle proteins. This approach enables facile and directed antibody selection of antibodies against all known four helix bundle proteins. Alternatively, these scaffolds may be engineered to contain multiple loops, single or multiple helical substitutions, or even combinations of loops and helices from any number of target proteins. By extension, other conserved structural elements of soluble and single span proteins could be utilized to present and direct antibody recognition to their critical elements within cognate superfamily proteins.

### Example 4

### Multispan Superfamily Conformational Antigens—GPCRs FIG. 1

Similarly to Example 3, single linear or discontinuous sequences of a target protein can be presented on a surrogate scaffold, such that the introduced sequence adopts partially, or completely, the conformational elements found in the native target protein. As an example, portions of the extracellular domains of multispan G-protein coupled receptors are grafted to an unrelated protein scaffold. The B1 fragment of protein G has a free amino terminus and a proximal loop structure that can be replaced with the mature amino terminus from CCR3 and the third extracellular loop from CCR3, respectively. In previous studies, the fusion bound the CCR3 cognate ligand, eotaxin and mutants, with similar ranked affinities to the full length CCR3 receptor, albeit with an overall 1000-fold reduction in affinities (Datta, Protein Sciences vol. 12, pg. 2482, Cold Spring Harbor Laboratory Press 2003). These results suggest that the soluble construct may provide sufficient conformational elements, in part, to mimic the receptor. For quality control binding to the CCR3, B1 chimera is tested with soluble or phage displayed eotaxin. Specifically, the amino terminus (amino acids 1 to 34) and the third extracellular loop of CCR3 (amino acids 265 to 281) are substituted for the amino terminal two amino acids and inserted between the loop amino acids 18 and 19 of the B1 domain fragment of Protein G. Next, this construct displayed on a spore surface is screened against a combinatorial antibody library through 3-5 rounds of enrichment. The enriched pools are then positively selected against an engineered mammalian cell line overexpressing CCR3. The resulting antibody clones are then recombinantly expressed, purified and then tested for binding to CCR3 or in eotaxin neutralization assay.

As a second example, the amino terminus of CCR2 and the third extracellular loop are similarly displayed on the B1 display scaffold and tested for reactivity to the neutralizing anti-CCR2 antibody 1D9. Specifically, the amino terminus (amino acids 1 to 42) and the third extracellular loop of CCR2 (amino acids 269 to 285) are substituted for the amino terminal two amino acids and between the loop amino acids 18 and 19 of the B1 domain fragment of Protein G. Next, this construct displayed on a spore surface is screened against a combinatorial antibody library through 3-5 rounds of enrichment. The enriched pools are positively selected against an engineered mammalian cell line overexpressing CCR2. The resulting clones are purified and tested for binding to CCR2 or in MCP-1 neutralization assays. Spore-phase screening is performed as described in Example 1.

Importantly, in these two examples, the third extracellular loop was selected, but the first or even the second extracellular loop may be used instead. It may be necessary or advantageous to incorporate more than one extracellular loop or even portions of the juxtamembrane regions from these loops to provide more conformational context. A similar approach can be applied to other proteins with structural motifs, such as all G-protein coupled receptors (GPCRs), ion channels, as well as other multispan and other soluble or integral multiloop proteins.

### Example 5

### Bioinformatic Approach to Identify and Generate Conformational Antigens

The previous examples describe methods using individual proteins or superfamilies, as well as random collections. Importantly, the random collections are likely derived from expressed cDNAs whose representation will be highly biased by relative expression levels and not limited to types of proteins or accessibility of fragments on the native proteins. Furthermore, in cloning random fragments one can, at best, only control directionality but not proper reading of the frame. Therefore, numerous unproductive clones are expected to be present that do not form a proper fusion at their amino or carboxy terminal ends. Some of these aspects can be addressed through a bioinformatic approach.

For instance, instead of directly cloning expressed gene fragments from activated lymphocytes as described in Example 1, gene expression in activated lymphocytes is examined to find genes of interest that have altered expression traits. As a primary filter, we could focus on those genes of interest that are extracellular. Next, the corresponding cDNAs are synthesized or rescued, and their fragments cloned similar to procedures described in the previous examples. As a result, only those fragments are produced that are in proper orientation and frame to result in productive fusions. The end result is that screening this collection yields only antibodies against extracellular proteins.

As an additional bioinformatic step, the previously described genes of interest are further examined and predicted solvated regions found on the outer surfaces of these proteins are identified. These solvated regions can then be synthesized and cloned into conformational scaffolds and screened against combinatorial antibody libraries. This additional step makes fragments only of predicted exposed regions of proteins, making the collection even more productive to accessible epitopes.

A major advantage of either synthetic approach is the resulting ability to normalize genes or even generate custom biases based upon other relevant criteria, such as gene induction levels or temporal expression. This approach can also be used on genes that encode predicted intracellular proteins and isolate antibodies for use as intrabodies.

### Example 6

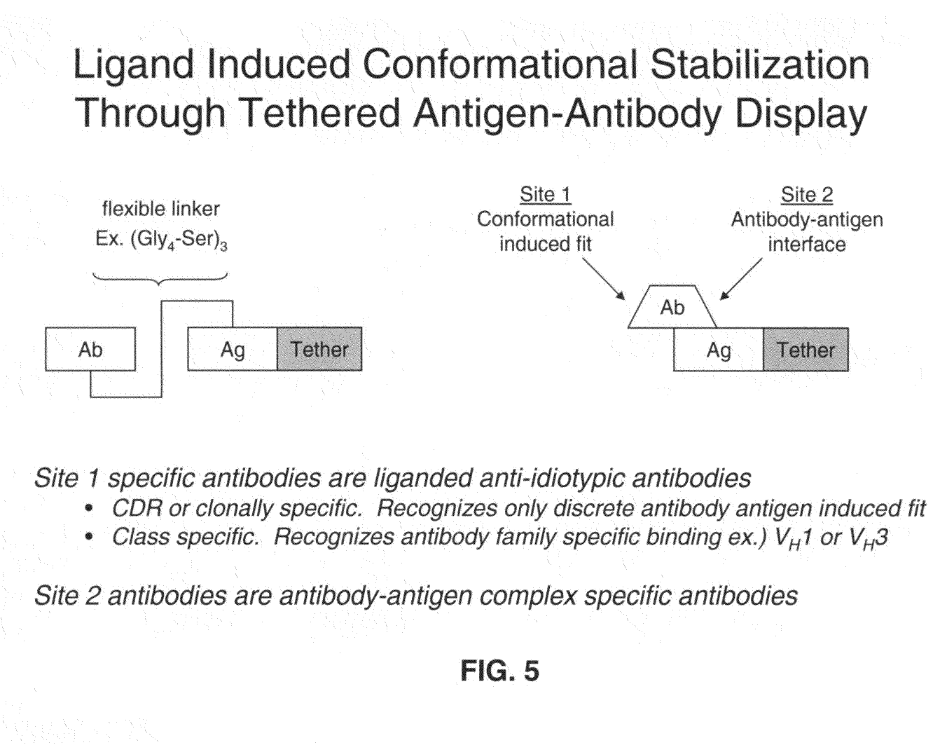

### Screening Antibody Antigen Complexes (FIG. 5)

Antibodies bind antigens through specific interactions in their variable regions. Stable binding is often formed and maintained due to induced fit, in either or both components. In the case of antibodies, it has been shown that liganded and unliganded antibodies are structurally distinct. This structural difference is exploited by using the conformational epitope presentation to screen for antibodies that recognize specific antibody-antigen complexes.

In one example, an antibody is cloned into the first site of the tandem expression plasmid and the cognate epitope cloned into the second site. Placing the antigen in close proximity to the antibody allows it to act as a conformational catalyst to the antibody. The result is a stable and tethered antigen-antibody complex suitable for complex screening. Additionally, because antigen binding induces unique conformational changes in the antibody, this stable presentation allows for efficient identification of liganded specific anti-idiotype antibodies.

For instance, an anti-c-myc antibody (9E10) could be expressed at the amino terminus of a flexible linker [(Gly₄-Ser)₃] and linked to the c-myc peptide epitope, which is recombinantly tethered to a bacillus protoxin. The resulting spore displayed complex is screened against a combinatorial phage antibody library to find anti-complex antibodies and anti-liganded anti-idiotype antibodies. Either of these resulting antibodies can be used as one arm of a bispecific antibody with the 9E10 antibody. Combining this bispecific antibody with an extracellularly decorated c-myc cell will have two consequences. The first is a “directed” reaction of the c-myc with the 9E10 arm. The second expected consequence is the “trans” reaction where the anti-idiotype arm binds a 9E10+c-myc protein complex, or the liganded 9E10 arm, of another bispecific antibody molecule. This “trans” reaction is a progressive reaction that continues until all target is stoichiometrically consumed. In practice, this “antibody chain reaction” decorates and reinforces binding of antibody to target, deliver, and stabilize maximal amounts of Fc to a target.

### Example 7

### Phage-Phage Target-Antibody Selection (FIG. 6)

In the previous examples, spores were used to display the epitope collection and phage to display the antibody selection. It is also possible to display both collections using the same display system, if the collections are both physically distinguishable for selection and genetically distinguishable for amplification.

In this example, the first step is to express the collections in plasmids with different antibiotic resistance, tetracycline resistance for the epitope library and ampicillin resistance for the antibody collection. Secondly, the phage coat proteins are epitope tagged to physically discern the discrete collections from each other. To do so, unique affinity tag(s) are recombinantly fused to the amino terminus of the pVII coat protein. Specifically, first either FLAG (DYKDDDDK) or HA (YPYDVPDYA) peptide pVII-tagged helper phage is generated. For instance, the HA helper phage can be used to generate the epitope library phagemid collection and the FLAG helper phage to produce the antibody phagemid library collection. 1e6-7 epitope library phagemid is mixed with 1e9-10 antibody library phagemid in one ml of PBS+1% BSA and incubated at room temperature for two hours. Next, anti-HA coated protein A beads are incubated with the mixture for one hour at 4° C. The beads are then washed 3-5 times with PBS+0.05% Tween-20. Finally, the beads are treated with pH 2.2 glycine for 10 minutes and then neutralized with 2M Tris-base. This step irreversibly dissociates the antibody-antigen complexes and allows them to infect E. coli and be amplified under either ampicillin or tetracycline selection. The amplified collections contain the entire epitope library and only productive antibody binders. The next selection step is similar to the previous step, but instead of HA precipitation, an anti-FLAG precipitation is performed. This step therefore removes all unbound epitope library members and reinforces only productive epitope-antibody collections.

Although this example cites antibody-antigen interaction, the same approach is expected to work for any number, or kind, of interactive phage-displayed collections. These can be antibodies against other antibodies, or even peptides against receptors or enzymes. It is also possible to accomplish similar results with a single immunologically distinct helper phage, if the tag is appropriately regenerated each round to the proper epitope or antibody population. This immunologically distinct difference can be engineered into a helper phage coat protein or may exist naturally between two distinct phage coat proteins.

### Example 8

### Simultaneous Selection of Conformation Epitope and Antibody Libraries

In the foregoing examples, the conformational epitope library collection was used to enrich antibodies that recognize native proteins or naturally presented targets. However, it is possible to perform simultaneous conformational target-antibody selection with the presently described system. The goal in this instance is to preserve specific target-antibody pairings. This is possible only when the target collection display is substantially and physically different from the antibody display collection, such as, for example, when the targets are associated with an insoluble spore particle and the antibodies are fused to a soluble filamentous bacteriophage. To preserve pairings, the target collections are first mixed with antibody collections. Following a suitable incubation period, the spore target collection is collected by centrifugation and unbound phage removed with appropriate washings. Following the wash steps to remove unbound phage, the complexed collections are incubated with monoclonal anti-phage antibodies and then combined with paramagnetic anti-mouse beads for positive magnetic selection of phage-bound spores. Alternatively, the phage-spore and anti-phage complexes can be detected with a fluorophore conjugated anti-mouse antibody, and individual spores can be clonally sorted by FACS. The paired combinations can be individually rescued under suitable conditions to addressably and independently rescue bacteriophage and propagate bacillus spores.

Although in the foregoing description the invention is illustrated with reference to certain embodiments, it is not so limited. Indeed, various modifications of the invention in addition to those shown and described herein will become apparent to those skilled in the art from the foregoing description and fall within the scope of the appended claims.

All references cited throughout the specification, and the references cited therein, are hereby expressly incorporated by reference in their entirety.

1. A physically selectable display comprising tandem or multimeric assemblies of discrete or random fragments of one or more native or variant polypeptides, or sequences mimicking said fragments, wherein at least some of said assemblies form conformationally constrained polypeptide targets, and wherein at least some of said fragments are other than antibody fragments.

2. The display of claim 1 which is a conformational epitope library.

3. The display of claim 1 comprising tandem or multimeric assemblies of discrete or random fragments of more than one polypeptide, or sequences mimicking said fragments.

4. The display of claim 1 wherein at least some of said tandem or multimeric assemblies comprise two or more fragments from different parts of the same polypeptide, or sequences mimicking said fragments.

5. The display of claim 1 wherein at least some of said tandem or multimeric assemblies comprise fragments from different polypeptides, or sequences mimicking said fragments. v

6. The display of claim 1 wherein at least some of said tandem or multimeric assemblies comprise an antibody or antibody fragment and a ligand for said antibody or antibody fragment.

7. The display of claim 1 wherein in said tandem or multimetic assemblies, at least some of said fragments or sequences are directly fused to each other.

8. The display of claim 1 wherein in said tandem or multimeric assemblies, at least some of said fragments or sequences are coupled by an exogenous connecting sequence.

9. The display of claim 1 wherein in said tandem or multimeric assemblies, at least some said fragments or sequences consist of or comprise a structural support element.

10. The display of claim 1 wherein at least some of the conformationally constrained polypeptide targets are formed as a result of the proximity of the fragments, or mimicking sequences, present in said tandem or multimeric assemblies.

11. The display of claim 1 wherein at least some of the conformationally constrained polypeptide targets are formed as a result of the presence of structural support elements in said tandem or multimeric assemblies.

12. The display of claim 11 wherein said structural support element is a motif characteristic of one or more protein families.

13. The display of claim 11 wherein said structural support element is selected from the group consisting of helical bundles, β-sheet structures, trifoil structures, membrane-spanning helices, and extracellular loops.

14. The display of claim 1 wherein the conformationally constrained polypeptide targets comprise receptor sequences.

15. The display of claim 14 wherein said receptor sequences include structural motifs of the receptors.

16. The display of claim 1 selected from the group consisting of in vivo and in vitro display systems.

17. The display system of claim 16 selected from the group of viral, eukaryotic, bacterial, ribosome, mRNA, and DNA display systems.

18. The display of claim 17 which is a bacteriophage display.

19. The display of claim 17 wherein said eukaryotic display system is a mammalian or yeast display.

20. The display of claim 17 wherein said bacterial display system is a bacterial cell or spore display.

21. The display of claim 20 wherein said bacterial display system is a Bacillus subtilis or Bacillus thuringiensis spore display.

22. The display of claim 11 wherein said bacterial display system is a Bacillus thuringiensis spore display.

23. A screening method, comprising
(a) providing a physically selectable display comprising tandem or multimeric assemblies of discrete or random fragments of one or more native or variant polypeptide, or sequences mimicking said fragments, wherein at least some of said assemblies form conformationally constrained polypeptide targets, and wherein at least some of said fragments are other than antibody fragments;
(b) contacting said display with a library of candidate binding partners under conditions wherein the conformationally constrained polypeptide targets and the candidate binding partners that have binding affinities to each other form target-binding partner complexes, and
(c) detecting at least some of the target-binding partner complexes formed.

24. The method of claim 23 further comprising the step of (d) identifying the target sequences participating in the formation of at least some of the target-binding partner complexes detected.

25. The method of claim 24 wherein the target sequences participating in the formation of all target-binding partner complexes detected are identified.

26. The method of claim 23 wherein said display comprises tandem or multimeric assemblies of discrete or random fragments of more than one polypeptide, or sequences mimicking said fragments.

27. The method of claim 23 wherein at least some of said tandem or multimeric assemblies comprise two or more sequences from different parts of the same polypeptide, or sequences mimicking said fragments.

28. The method of claim 23 wherein at least some of said tandem or multimeric assemblies comprise fragments from different polypeptides, or sequences mimicking said fragments.

29. The method of claim 23 wherein at least some of said tandem or multimeric assemblies comprise an antibody or antibody fragment and a ligand for said antibody or antibody fragment.

30. The method of claim 23 wherein in said tandem or multimetic assemblies, at least some of said fragments or sequences are directly fused to each other.

31. The method of claim 23 wherein in said tandem or multimeric assemblies, at least some of said fragments or sequences are coupled by an exogenous connecting sequence.

32. The method of claim 23 wherein in said tandem or multimeric assemblies, at least some of the two or more sequences consist of or comprise a structural support element.

33. The method of claim 23 wherein at least some of the conformationally constrained polypeptide targets are formed as a result of the proximity of the fragments, or mimicking sequences, present in said tandem or multimeric assemblies.

34. The method of claim 23 wherein at least some of the conformationally constrained polypeptide targets are formed as a result of the presence of structural support elements in said tandem or multimeric assemblies.

35. The method of claim 34 wherein said structural support element is a motif characteristic of one or more protein families.

36. The method of claim 34 wherein said structural support element is selected from the group consisting of helical bundles, β-sheet structures, trifoil structures, a membrane-spanning helices, and extracellular loops.

37. The method of claim 23 wherein the candidate binding partners are antibodies or antibody fragments.

38. The method of claim 37 wherein said antibody fragments are selected from the group consisting of Fab, Fab′, F(ab′)₂, dAb, scFv and (scFv)₂ fragments, linear antibodies, single-chain antibody molecules, minibodies, diabodies, and multispecific antibodies formed from antibody fragments.

39. The method of claim 38 wherein said antibody fragments are scFv fragments.

40. The method of claim 37 wherein said antibodies or antibody fragments are part of an antibody library.

41. The method of claim 23 wherein the candidate binding proteins are antibody mimics.

42. The method of claim 41 wherein the antibody mimics are affibodies or aptamers.

43. The method of claim 23, wherein said physically selectable display is an in vivo or in vitro display system.

44. The method of claim 43, wherein said physically selectable display is selected from the group consisting of viral, eukaryotic, bacterial, ribosome, mRNA, and DNA display systems.

45. The method of claim 44 wherein said display system is a bacteriophage display.

46. The method of claim 44 wherein said eukaryotic display system is a mammalian or yeast display.

47. The library of claim 44 wherein said bacterial display system is a bacterial cell or spore display.

48. The method of claim 47 wherein said bacterial display system is a Bacillus subtilis or Bacillus thuringiensis spore display.

49. The method of claim 40 wherein said antibody library is displayed.

50. The method of claim 49 wherein the antibody display is an in vivo or in vitro display system.

51. The method of claim 49 wherein the antibody display is selected from the group consisting of viral, eukaryotic and bacterial display systems.

52. The method of claim 51 wherein said display system is a bacteriophage display.

53. The method of claim 51 wherein said eukaryotic display system is a mammalian or yeast display.

54. The library of claim 51 wherein said bacterial display system is a bacterial cell or spore display.

55. The method of claim 54 wherein said bacterial display system is a Bacillus subtilis or Bacillus thuringiensis spore display.

56. The method of claim 49 wherein the antibody library is a phage library, and the physically selectable display is a spore display or a phage display.

57. The method of claim 56 wherein the spore display is a Bacillus thuringiensis spore display.

58. The method of claim 23 wherein the conformationally constrained polypeptide targets comprise receptor sequences.

59. The method of claim 58 wherein the binding partners are ligand candidates for the receptors.

60. The method of claim 59 wherein said receptor sequences include structural motifs of the receptors.

61. The method of claim 38 wherein the antibody or antibody fragment sequences participating in the formation of at least some of the target-binding partner complexes are additionally identified.

62. The method of claim 61 further comprising the step of enriching and segregating the target sequences and the antibody sequences participating in the formation of at least some of the target-binding partner complexes prior to step (d).

63. The method of claim 62 further comprising the step of independently recovering the target sequences and the antibody sequences participating in the formation of at least some of the target-binding partner complexes following the enrichment and segregation and prior to step (d).

64. The method of claim 38 wherein the target sequences participating in the formation of at least some of the target-binding partner complexes are parts of a conformational epitope.

65. A method, comprising
(a) providing a physically selectable display comprising tandem or multimeric assemblies of discrete or random fragments of one or more native or variant polypeptide, or sequences mimicking said fragments, wherein at least some of said assemblies form conformational epitopes;
(b) contacting said display with an antibody library under conditions wherein the conformational epitopes and members of the antibody library that have binding affinities to each other form conformational epitope-antibody complexes; and
(c) detecting at least some of the conformational epitope-antibody complexes formed.

66. The method of claim 65 further comprising the step of (d) identifying the conformational epitope and antibody sequences participating in the formation of at least some of the conformational epitope-antibody complexes detected.

67. The method of claim 66 wherein all conformational epitope-antibody complexes formed are detected.

68. The method of claim 67 wherein the conformational epitope sequences participating in the formation of all target-binding partner complexes detected are identified.

69. The method of claim 65 wherein said display comprises tandem or multimeric assemblies of discrete or random fragments of more than one polypeptide, or sequences mimicking said fragments.

70. The method of claim 65 wherein at least some of said tandem or multimeric assemblies comprise fragments from different parts of the same polypeptide, or sequences mimicking said fragments.

71. The method of claim 65 wherein at least some of said tandem or multimeric assemblies comprise fragments from different polypeptides, or sequences mimicking said fragments.

72. The method of claim 71 wherein at least some of said tandem or multimeric assemblies comprise an antibody or antibody fragment and a ligand for said antibody or antibody fragment.

73. The method of claim 65 wherein in said tandem or multimetic assemblies, at least some of said fragments or sequences are directly fused to each other.

74. The method of claim 65 wherein in said tandem or multimeric assemblies, at least some of said fragments or sequences are coupled by an exogenous connecting sequence.

75. The method of claim 65 wherein in said tandem or multimeric assemblies, at least some of said fragments or sequences consist of or comprise a structural support element.

76. The method of claim 65 wherein at least some of the conformational epitopes are formed as a result of the proximity of the fragments, or mimicking sequences, present in said tandem or multimeric assemblies.

77. The method of claim 65 wherein at least some of the conformational epitopes are formed as a result of the presence of structural support elements in said tandem or multimeric assemblies.

78. The method of claim 77 wherein said structural support element is a motif characteristic of one or more protein families.

79. The method of claim 77 wherein said structural support element is selected from the group consisting of helical bundles, β-sheet structures, trifoil structures, membrane-spanning helices, and extracellular loops.

80. The method of claim 65 wherein said antibody library comprises antibody fragments.

81. The method of claim 80 wherein said antibody fragments are selected from the group consisting of Fab, Fab′, F(ab′)₂, dAb, scFv, and (scFv)₂ fragments, linear antibodies, single-chain antibody molecules, minibodies, diabodies, and multispecific antibodies formed from antibody fragments.

82. The method of claim 81 wherein said antibody fragments are scFv fragments.

83. The method of claim 65 wherein said physically selectable display is a bacterial cell or spore display.

84. The method of claim 83 wherein said bacterial display system is a Bacillus subtilis or Bacillus thuringiensis spore display.

85. The method of claim 65 wherein the antibody library is a phage library, and the physically selectable display is a spore display or a phage display.

86. The method of claim 85 wherein the spore display is a Bacillus thuringiensis spore display.

87. The method of claim 65 wherein the conformational epitopes are obtained by the expression of tandem or multimeric assemblies of gene fragments.

88. The method of claim 87 wherein the gene fragments originate from a targeted, biologically relevant source.

89. The method of claim 88 wherein said targeted biologically relevant source is selected from the group consisting of cells, tissues, organs and organisms.

90. The method of claim 89 wherein said targeted biologically relevant source is selected from the group consisting of stem cells, activated immune cells, diseased tissues, organs and pathological organisms.

91. The method of claim 88 wherein at least some of the gene fragments are identified by analysis of gene expression data in a targeted, biologically relevant source.

92. A nucleic acid molecule comprising nucleotide sequences encoding an antibody or an antibody fragment and a ligand of said antibody or antibody fragment, separated by a nucleotide sequence encoding a peptide linker.

93. A vector comprising the nucleic acid molecule of claim 92.

94. A host cell transformed with the vector of claim 93.

95. The host cell of claim 94 which is an eukaryotic or prokaryotic host cell.

96. The host cell of claim 95 which is a bacterial, mammalian or yeast cell.

97. The host cell of claim 95, which is an E. coli cell.
